(19) United States
(12) Patent Application Publication
(10) Pub. No.: US 2008/0249599 A1
(43) Pub. Date: Oct. 9, 2008

(54) Stent With Therapeutic Agent Delivery Structures in Low Strain Regions

(75) Inventors: Jeffrey Allen, Santa Rosa, CA (US); Scott Doig, Santa Rosa, CA (US); Matthew J. Birdsall, Santa Rosa, CA (US); Darrel Untereker, Oak Grove, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/696,816

(22) Filed: Apr. 5, 2007

(51) Int. Cl. A61F 2/82

(52) U.S. Cl. 623/1.4; 623/1.42; 977/781

(57) ABSTRACT

A system for treating abnormalities of the cardiovascular system includes a stent having a plurality of therapeutic agent-carrying regions and non therapeutic agent-carrying regions. The therapeutic agent-carrying regions are located within low strain regions of the stent and the non therapeutic agent-carrying regions are located within high strain regions of the stent. Another embodiment of the invention includes a method of manufacturing a therapeutic agent-carrying stent comprising forming a stent framework and applying a formulation containing one or more therapeutic agents to the stent framework while preventing the therapeutic agents from contacting the high strain regions of the stent framework.

### TECHNICAL FIELD

This invention relates generally to biomedical devices that are used for treating vascular conditions. More specifically, the invention relates to a therapeutic agent eluting stent having one or more therapeutic agent eluting portion localized in low strain regions of the stent.

### BACKGROUND OF THE INVENTION

Stents are generally cylindrical-shaped devices that are radially expandable to hold open a segment of a vessel or other anatomical lumen after implantation into the body lumen.

Various types of stents are in use, including expandable and self-expanding stents. Expandable stents generally are conveyed to the area to be treated on balloon catheters or other expandable devices. For insertion into the body, the stent is positioned in a compressed configuration on the delivery device. For example, the stent may be crimped onto a balloon that is folded or otherwise wrapped about the distal portion of a catheter body that is part of the delivery device. After the stent is positioned across the lesion, it is expanded by the delivery device, causing the diameter of the stent to expand. For a self-expanding stent, commonly a sheath is retracted, allowing the stent to expand.

Stents are used in conjunction with balloon catheters in a variety of medical therapeutic applications, including intravascular angioplasty. For example, a balloon catheter device is inflated during percutaneous transluminal coronary angioplasty (PTCA) to dilate a stenotic blood vessel. The stenosis may be the result of a lesion such as a plaque or thrombus. When inflated, the pressurized balloon exerts a compressive force on the lesion, thereby increasing the inner diameter of the affected vessel. The increased interior vessel diameter facilitates improved blood flow. Soon after the procedure, however, a significant proportion of treated vessels restenose.

To reduce restenosis, stents, constructed of metals or polymers, are implanted within the vessel to maintain lumen size. The stent is sufficiently longitudinally flexible so that it can be transported through the cardiovascular system. In addition, the stent requires sufficient radial strength to enable it to act as a scaffold and support the lumen wall in a circular, open configuration. Configurations of stents include a helical coil, and a cylindrical sleeve defined by a mesh, which may be supported by a stent framework of struts or a series of rings fastened together by linear connecter portions.

Stent insertion may cause undesirable reactions such as inflammation resulting from a foreign body reaction, infection, thrombosis, and proliferation of cell growth that occludes the blood vessel. Stents capable of delivering one or more therapeutic agents have been used to treat the damaged vessel and reduce the incidence of deleterious conditions including thrombosis and and restenosis.

Polymer coatings applied to the surface of the stents have been used to deliver drugs or other therapeutic agents at the placement site of the stent. The coating is sometimes damaged during expansion of the stent at the delivery site, causing the coating to chip off the stent and release flakes of the polymer coating, which reduces the effective dose of the drug at the treatment site, and under some circumstances, may result in emboli in the microvasculature.

Recently, stents have been introduced that have a porous surface, usually consisting of indentations in the surface of the stent. The indentations can be filled with a formulation containing drugs or other therapeutic agents that will leach from the stent after it is deployed, without a polymer coating covering the external surface of the stent. One drawback of stents comprising porous materials, however, is that the structure of the stent is weakened compared to a stent structure of solid metal or polymer. Consequently, a porous stent may crack or break during expansion at the treatment site as a result of the strain placed on certain regions of the stent.

Strain is a measure of the displacement that can be applied to a material before the material breaks or tears. Strain is measured as the ratio of the change in length of the material to the original length of the material.

Clearly, the strain applied to the various regions of the stent framework during delivery and deployment of the stent is a parameter that must be considered in stent design. FIGS. 1A and 1B portray stent framework 100, which comprises a series of elongated strut portions 102 and curved crown portions 104, longitudinally adjoining strut portions 102. Strut portions 102 provide radial strength, enabling stent framework 100 to support the vessel wall and maintain patency. Crown portions 104 act as flexible hinges allowing the angles formed by crown portions 104 to increase or decrease as stent framework 100 expands or contracts. The bending of crown portions 104 to a wider or narrower angle places crown portions 104 under strain.

FIG. 1A shows the configuration of stent framework 100 when the stent is compressed, as for example when it is mounted on a catheter during delivery. The stent has a reduced diameter and stent framework 100 is in a compressed configuration, with crown portions 104 acutely bent, and struts 102 approximately parallel to each other. When the stent is deployed at the treatment site, the stent is expanded. As the diameter of the stent increases, struts 102 move laterally away from each other and the angle formed by crown portion 104 is enlarged as shown in FIG. 1B. The opening and closing of the angle formed by crown portions 104 causes significant strain on crown portions 104. In contrast, little strain is placed on strut portions 102 of the stent.

FIGS. 2A and 2B are a schematic representation of stent 200 comprising a mesh overlaying stent framework 100. To form stent 200, shown in FIGS. 2A and 2B, the flat planar configuration of stent framework 100 shown in FIG. 1A, is formed into the cylindrical or tubular structure shown in FIG. 2A. Strut portions 102 provide radial strength, enabling stent 200 to maintain vessel patency.

For delivery, vascular stents are frequently mounted on a delivery catheter in a compressed configuration as shown in FIGS. 1A and 2A, and transported through the vascular system to the site of the vascular lesion requiring treatment. Once at the treatment site, stent 200 is deployed from the catheter by radially expanding stent 200, and lodging stent 200 firmly against the interior surface of the vascular wall. Stent framework 100 is shown in an expanded configuration in FIGS. 1B and 2B. As shown in FIG. 1B, strut portions 102 move laterally away from each other as the diameter of stent 200 increases (FIG. 2B). Stent 200 may be self-expanding or balloon expandable, depending on both the dimensions of stent 100 and the material comprising stent 100.

FIGS. 3A and 3B are schematic representations of stent framework 300 comprising a series of parallel, expandable rings 304 held together by linear connector struts 302. Shown in FIG. 3A is the contracted configuration of stent framework 300 in which the parallel rings are compressed to a smaller diameter than in the expanded form of the stent shown in FIG. 3B. As stent framework 300 expands and contracts, rings 304 increase and decrease in diameter, placing strain on the material comprising rings 304. In contrast, little or no strain is placed on linear connectors 302.

Regardless of stent configuration, experience has shown that chipping of a coating during delivery of a stent occurs in high strain areas of the stent due to the movement of the stent framework and the strain placed on the stent within these areas. In addition, the stent is most likely to crack or break in the high strain areas as the material comprising the stent framework is not strong and flexible enough to withstand the strain placed on these areas during expansion and contraction of the stent.

It would be desirable, to provide an implantable drug eluting stent that retains the lateral flexibility needed for delivery and deployment and the radial strength to support the vessel wall, but also exhibits minimal chipping and flaking of the drug/polymer coating, or cracking of the stent body in the case of porous stents, when the stent is stressed during delivery and deployment. Such a stent would overcome many of the limitations and disadvantages inherent in the devices described above.

### SUMMARY OF THE INVENTION

One aspect of the present invention provides a system for treating abnormalities of the cardiovascular system comprising a catheter and a therapeutic agent-carrying stent disposed on the catheter. The stent includes a stent framework having plurality of therapeutic agent-carrying regions and non therapeutic agent-carrying regions. The therapeutic agent-carrying regions are disposed within regions of the stent that are subjected to low strain when the stent is expanded or contracted, and the non therapeutic agent-carrying regions are disposed within regions of the stent framework that are subjected to high strain when the stent is expanded or contracted.

Another aspect of the invention provides an expandable stent comprising a stent framework having a plurality of regions subjected to high strain during expansion or contraction of the stent and a plurality of regions subjected to low strain during expansion or contraction. Further, the stent has therapeutic agent-carrying regions localized within the low strain regions and non therapeutic agent-carrying regions localized within the high strain regions.

Another aspect of the invention provides a method for manufacturing a therapeutic agent-carrying stent having high and low strain regions during expansion and contraction of the stent. First, the stent framework is formed. Next, a formulation containing one or more therapeutic agents is disposed on the low strain regions of the stent framework, without contacting the high strain areas of the stent framework with the formulation.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1A is a schematic illustration of a portion of a strut and crown stent framework in a compressed configuration;

FIG. 1B is a schematic illustration of a portion of a strut and crown stent framework in an expanded configuration;

FIG. 2A is an exterior view of a cylindrical stent when the stent is compressed;

FIG. 2B is an exterior view of a cylindrical stent when the stent is expanded;

FIG. 3A is a schematic illustration of a stent framework comprising rings and longitudinal connecters in a compressed configuration;

FIG. 3B is a schematic illustration of an expanded configuration of a stent framework comprising rings and longitudinal connectors indicating the regions of the stent framework that are subjected to strain during expansion and contraction;

FIG. 4 is a schematic illustration of the stent framework shown in FIG. 1B indicating the therapeutic and non therapeutic agent-carrying regions of the stent undergoing increased strain due to radial expansion of the stent, in accordance with the present invention;

FIG. 5 is a schematic illustration of the stent framework shown in FIG. 3B indicating the therapeutic and non therapeutic agent-carrying regions of the stent, in accordance with the present invention;

FIG. 6 is a flow diagram of a method of manufacturing an expandable stent with therapeutic agent-carrying and non therapeutic agent-carrying regions in low and high strain regions, respectively, in accordance with the present invention.

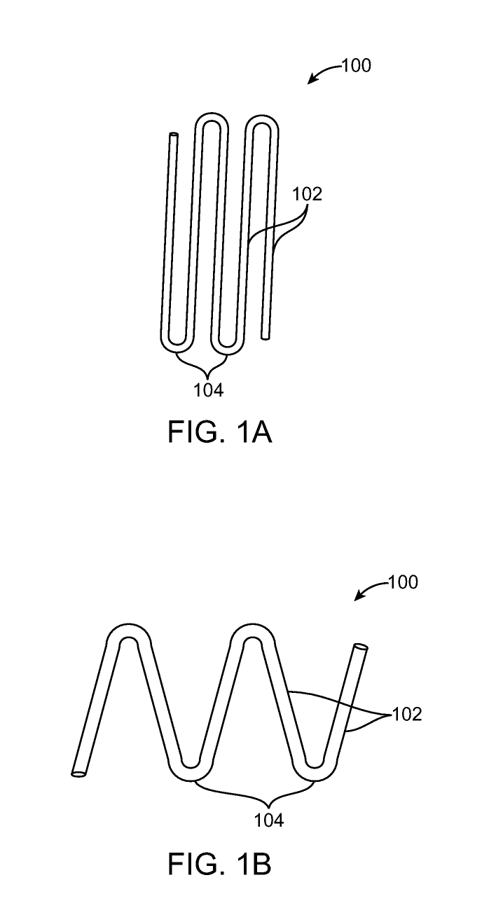

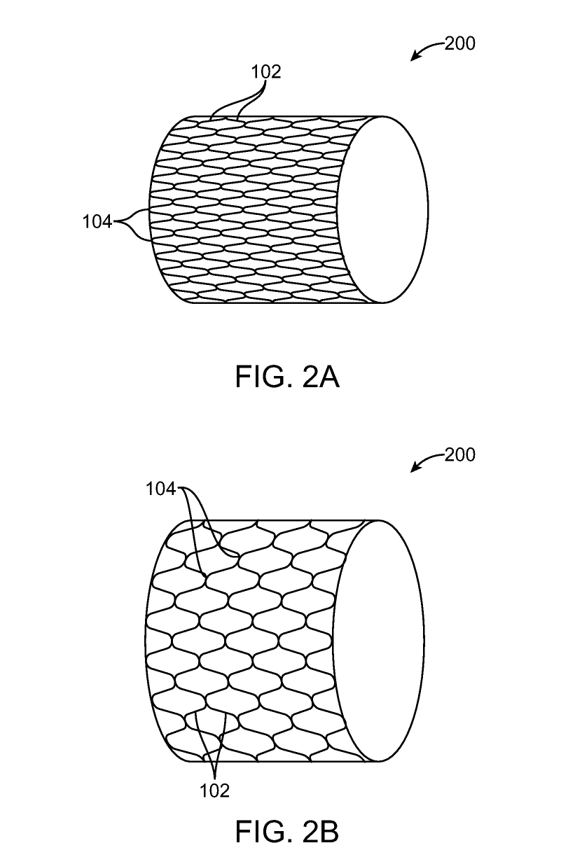

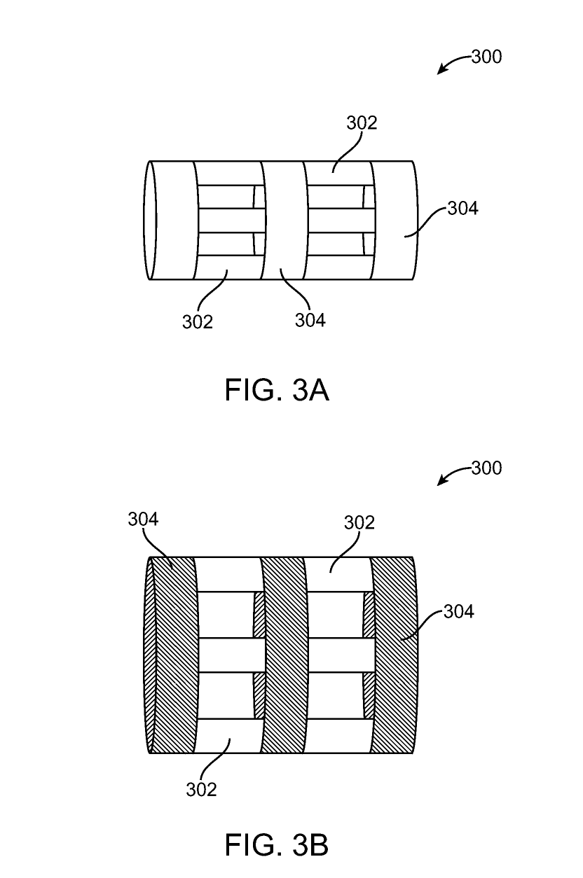

### DETAILED DESCRIPTION

Throughout this specification, like numbers refer to like structures.

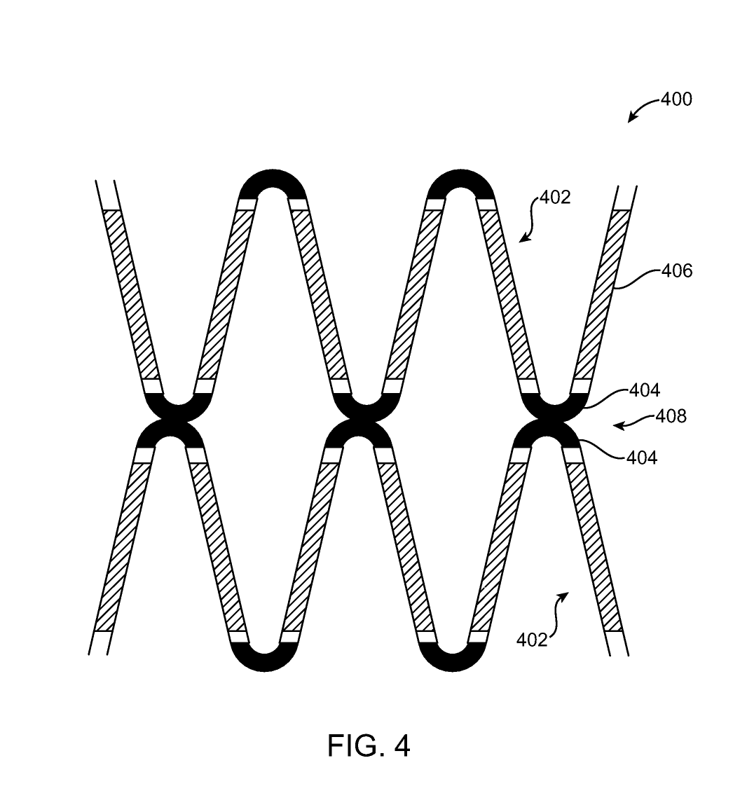

Referring to the figures, FIG. 4 is a schematic representation of stent framework 400 having a strut and crown configuration with therapeutic agent carrying regions only in low strain areas 406 of stent framework 400. As the stent expands or contracts, crown portions 404 act as hinges, and the angle formed by the crown portion of the framework increases or decreases respectively. Each crown portion 404 is strained, causing high strain region 408 in stent framework 400. In contrast, relatively little strain is placed on strut portions 402 making strut portions 402 low strain regions within the stent framework. There is however, a gradient of strain between high strain regions 408 and low strain strut regions 402. In one embodiment of the invention, therapeutic agent-carrying regions 406 are located within low strain, strut portions 402.

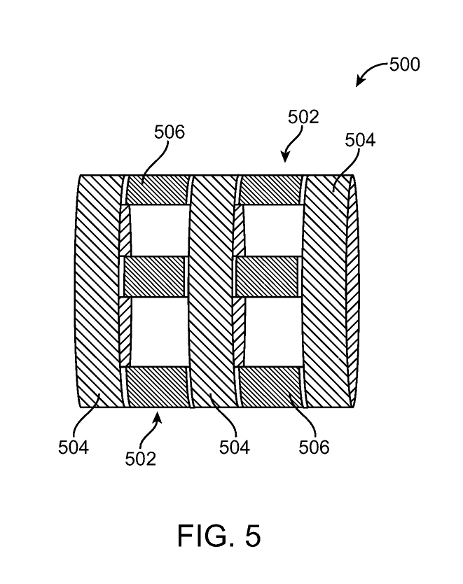

FIG. 5 is a schematic representation of stent framework 500 comprising a series of parallel, expandable ring portions 504 held together by longitudinal connector strut portions 502. As stent framework 500 expands or contracts, ring portions 504 increase or decrease respectively in diameter, causing strain to be placed on ring portions 504 in proportion to the amount of increase or decrease in diameter. However, if the diameter of each of ring portions 504 is increased or decreased by the same amount, little strain is placed on longitudinal strut portions 502, making longitudinal strut portions 502 low strain regions within the stent framework. There is, however, a transitional gradient of strain at the junctions of connector strut portions 502 and ring portions 504. In one embodiment of the invention, therapeutic agent-carrying regions 506 are located within longitudinal strut portions 502.

In one embodiment of the invention, the stent framework, such as stent frameworks 400 and 500, comprises one or more of a variety of biocompatible metals including stainless steel, titanium, gold, nickel/titanium alloys, such as nitinol, platinum, and platinum-tungsten alloys. These metallic materials are sufficiently flexible to allow the stent framework to be compressed and expanded, but also provide sufficient radial strength to maintain the stent in the expanded configuration, and apply adequate force to the vessel wall to hold the stent in place and maintain vessel patency.

In another embodiment of the invention, the stent framework comprises one or more biocompatible polymeric or metallic materials. Polymeric stents may be biodegradable, biostable, or comprise a mixture of polymeric materials that are both biostable and biodegradable. Biodegradable polymers appropriate for the stents of the invention include polylactic acid, polyglycolic acid, and their copolymers, caproic acid, polyethylene glycol, polyanhydrides, polyacetates, polycaprolactones, poly(orthoesters), polyamides, polyurethanes and other suitable polymers. Biostable polymers appropriate for the stents of the invention include polyethylene, polypropylene, polymethyl methacrylate, polyesters, polyamides, polyurethanes, polytetrafluoroethylene (PTFE), polyvinyl alcohol, and other suitable polymers. These polymers may be used alone or in various combinations to give the stent unique properties such as controlled rates of degradation, or to form biostable stents with a biodegradable or bioerodable coating that may reduce inflammation, control tissue ingrowth, and additionally, release one or more therapeutic agents. Alternatively, either a metallic or polymeric stent may be coated with a porous metal or metal oxide coating.

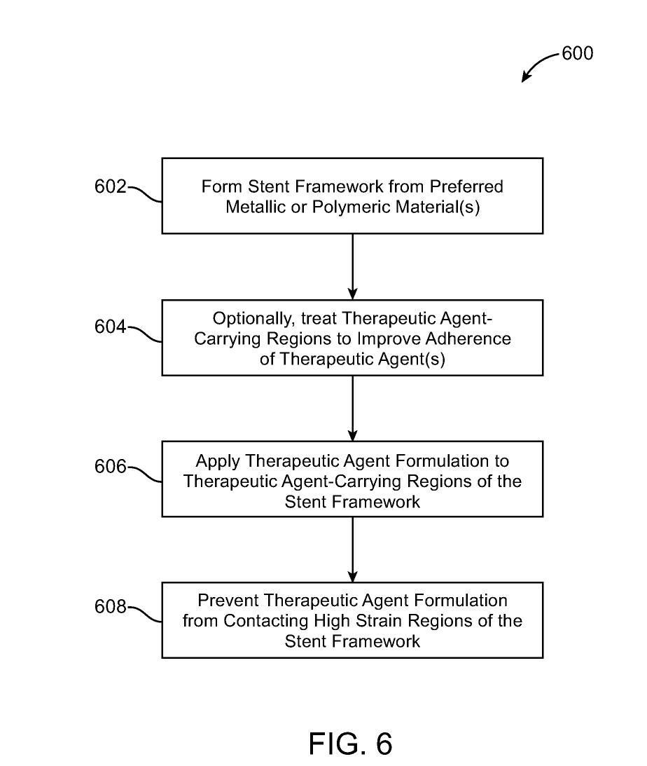

FIG. 6 is a flowchart of method 600 for manufacturing a therapeutic agent eluting stent in accordance with the present invention. The method includes forming a stent framework such as either stent framework 400 or 500, as indicated in Block 602. In some embodiments, a metallic wire is formed into a tubular shape about a mandrel. Alternatively, a sheet of metallic or polymeric material is laser cut and rolled into a tubular shape to form the stent framework. Using either method, a tubular stent framework is formed having a manufactured diameter that is intermediate between the diameter of stent framework in the compressed and the expanded configurations.

In one embodiment of the invention, therapeutic agent carrying regions 406 or 506 are treated to improve adherence of one of more therapeutic agents, as indicated in Block 604. Such treatment includes etching or pitting the surface of regions 406 or 506, or applying a primer polymeric coating or other appropriate methods. In other embodiments, the surface is chemically etched. Any such process is applied only to therapeutic agent carrying regions 406 or 506, and not to high strain regions 408 or 504.

Next, as indicated in Block 606, a formulation containing one or more therapeutic agents is applied to therapeutic agent-carrying regions 406 or 506 of stent framework 400 or 500 while preventing the formulation from contacting high strain regions 408 or 504 (Block 608). The therapeutic agent containing formulation may be applied to regions 406 or 506 by spraying or dipping stent framework 400 or 500 while shielding high strain regions 408 or 504 if needed. The framework is masked in one embodiment. Alternatively, an ink jet sprayer, in one embodiment, selectively applies the formulation to the stent framework.

Finally, the manufacture of the stent is completed by drying or curing the therapeutic agent formulation and adding a mesh over the exterior surface of stent framework 400 or 500, or any other procedure required by the design of the stent. The completed stent may then be compressed and mounted on a catheter, expanded at the delivery site, and otherwise handled as needed with minimal chipping, flaking, and loss of the therapeutic agent.

In one embodiment of the invention, a stent framework such as either stent framework 400 or 500 is formed from one or more metallic or polymeric materials. Next, the therapeutic agent-carrying region (406 or 506) of stent framework 400 or 500 is treated to improve adherence of one or more therapeutic agents. In one embodiment of the invention, cavities are created in the surface of the stent framework by processes such as abrasion, chemical etching, chemical dealloying, thermal dealloying, laser drilling, ion beam irradiation or any other appropriate method. The high strain regions of the stent framework, for example crown portions 404 of stent framework 400 or ring portions 504 of stent framework 500, are left unaltered. This is accomplished by directing the process only at the low strain regions of the stent framework, and if needed, additionally shielding the high strain regions. The cavities formed in the therapeutic agent carrying region are then filled with a formulation appropriate for the therapeutic agent(s) to be delivered. In one embodiment, the cavities are pores. In another embodiment, the cavities are nanopores with a diameter of less than about 500 nanometers.

In another embodiment of the invention, cavities are formed in low strain regions 402 and 502 as described above. However, in this embodiment, a gradient of decreasing density of cavities is formed in the regions of stent framework 400 or 500 approaching high strain regions 408 or 504. This design allows maximal therapeutic agent-carrying regions, while providing sufficient strength through the transition areas to prevent stent framework 400 or 500 from cracking or breaking.

In yet another embodiment, at least one region of a metallic wire is treated to increase porosity, interspersed with untreated regions. A stent framework such as stent framework 400 is then formed so that the treated regions become therapeutic agent-carrying regions 406 and untreated regions become high strain regions 408.

In one embodiment of the invention, various therapeutic agents, such as anticoagulants, antiinflammatories, fibrinolytics, antiproliferatives, antibiotics, therapeutic proteins or peptides, recombinant DNA products, or other bioactive agents, diagnostic agents, radioactive isotopes, or radiopaque substances are applied to the therapeutic agent-carrying region of the stent. The formulation containing the therapeutic agent may additionally contain excipients including solvents or other solubilizers, stabilizers, suspending agents, antioxidants, and preservatives, as needed to deliver an effective dose of the therapeutic agent to the treatment site. In some embodiments of the invention, the formulation is applied as a liquid to the therapeutic agent-carrying region of the stent framework so that the porous structures are filled with the formulation. The formulation is then dried to remove the solvent using air, vacuum, or heat, and any other effective means of causing the formulation to adhere to the stent framework.

In another embodiment of the invention one or more therapeutic agents are deposited on therapeutic agent-carrying region 406 or 506 of stent framework 400 or 500 in a coating applied to the external surface of stent framework 400 or 500. In another embodiment, the therapeutic agent(s) are deposited on the surface of therapeutic agent-carrying region 406 or 506 of stent framework 400 or 500, and then a polymeric or non-polymeric coating is applied over the therapeutic agents. In some embodiments the coating includes one or more polymers that optimize the delivery and availability of the therapeutic agent.

In one embodiment of the invention, the coating material is deposited on the surface of low strain regions 402 or 502 of stent framework 400 or 500 by rotating stent framework 400 or 500 and spraying the coating material from a nozzle that selectively directs the spray at low strain regions 406 or 506 of stent framework 400 or 500 while leaving high strain regions 408 or 504 unaffected. In some embodiments, the coating is then dried using air, vacuum, or heat, or is cured using ultraviolet light causing the coating to adhere to the surface of low strain region 402 or 502 of stent framework 400 or 500. In one embodiment, the nozzle is a portion of an ink jet printing device.

In still other embodiments of the invention, high strain regions 408 or 504 of stent framework 400 or 500 are shielded while the formulation containing the therapeutic agent is applied to low strain regions 402 or 502. For example, high strain regions 408 or 504 of stent framework 400 or 500 may be covered with a physical barrier while the therapeutic agent-containing formulation is applied to low strain regions 402 or 502 of stent framework 400 or 500. In another embodiment, high strain regions 408 or 504 may be coated with a polymer solution or oil before the therapeutic agent formulation is applied to stent framework 400 or 500 by dipping or spraying so that the formulation will not adhere to high strain regions 408 or 504.

While the invention has been described with reference to particular embodiments, it will be understood by one skilled in the art that variations and modifications may be made in form and detail without departing from the spirit and scope of the invention.

1. A system for treating a vascular condition comprising:
a catheter;
a therapeutic agent-carrying stent disposed on the catheter, the stent including a stent framework having a plurality of therapeutic agent-carrying regions and non therapeutic agent-carrying regions wherein the therapeutic agent-carrying regions are disposed within low strain regions of the stent, the non therapeutic agent-carrying regions are disposed within high strain regions of the stent, and the therapeutic agent-carrying regions have a first level of porosity, the non therapeutic agent-carrying regions have a second level of porosity, and the first level of porosity is greater than the second level of porosity.

2. The system of claim 1 wherein the stent framework is expandable, and the high strain regions comprise crown portions of the stent framework, the low strain regions comprise elongated strut portions of the stent framework extending from the crown portions in a longitudinal, planar relationship, and the therapeutic agent carrying regions are disposed within the strut regions.

3. The system of claim 1 further comprising a decreasing gradient of porosity through the region of the stent framework between the first level of porosity and the second level of porosity

4. The system of claim 1 wherein the stent is an expandable stent and the low strain regions comprise connector portions of the stent and the high strain regions comprise ring portions of the stent, and the therapeutic agent-carrying regions are disposed within the connector portions of the stent.

5. The system of claim 1 wherein the pores are nanopores having a diameter less than or equal to 5 nm.

6. The system of claim 1 wherein wherein the stent framework comprises one or more biocompatible materials selected from the group consisting of metals, metal alloys, biostable polymers, biodegradable polymers, bioerodible polymers and other suitable materials.

7. The system of claim 1 wherein one or more therapeutic agents are applied to the therapeutic agent-carrying regions of the stent using one or more coatings selected from the group consisting of polymeric coatings, non-polymeric coatings, and porous metal coatings.

8. The system of claim 7 wherein at least one therapeutic agent is contained in the coating.

9. The system of claim 7 wherein the therapeutic agent is disposed on the therapeutic agent-carrying regions of the stent and subsequently the coating is placed over the therapeutic agent.

10. The system of claim 1 wherein the porosity of the low strain regions of the stent is formed by subjecting the low strain regions to one or more processes selected from the group consisting of abrasion, chemical dealloying, thermal dealloying, ion beam irradiation, and laser drilling.

11. The system of claim 10 further comprising forming a decreasing gradient of porosity in the region of the stent framework between the first level of porosity and the second level of porosity.

12. An expandable therapeutic agent-carrying stent comprising a stent framework having a plurality of therapeutic agent-carrying regions and non therapeutic agent-carrying regions wherein the therapeutic agent-carrying regions are disposed within low strain regions of the stent and the non therapeutic agent-carrying regions are disposed within high strain regions of the stent, and the therapeutic agent-carrying regions have a first level of porosity, the non therapeutic agent-carrying regions have a second level of porosity, and the first level of porosity is greater than the second level of porosity.

13. The stent of claim 12 further comprising a decreasing gradient of porosity through the region of the stent framework between the first level of porosity and the second level of porosity.

14. The stent of claim 12 wherein the high strain regions of the stent framework comprise crown portions and the low strain regions comprise elongated strut portions of the stent framework extending from the crown portions in a longitudinal, planar relationship, and the therapeutic agent carrying regions are disposed within the strut regions.

15. The stent of claim 12 wherein the high strain regions of the stent framework comprise expandable ring portions of the stent framework and the low strain regions of the stent framework comprise connector portions, and the therapeutic agent-carrying regions are disposed within the connector portions of the stent.

16. A method of manufacturing a therapeutic agent-carrying stent framework comprising:
forming the stent framework having high strain regions and low strain regions;
forming a first level of porosity within the low strain regions and forming a second level of porosity within the high strain regions wherein the first level of porosity is greater than the second level of porosity; and
disposing a formulation containing one or more therapeutic agents to the low strain regions of the stent framework and preventing the therapeutic agents from contacting the high strain regions.

17. The method of claim 15 further comprising disposing the therapeutic agent on the surface of the low strain region of the stent by air drying to remove one or more solvents from the formulation containing the therapeutic agents.

18. The method of claim 15 further comprising forming pores in the surface of the low strain regions by subjecting the low strain regions to one or more processes selected from the group consisting of abrasion, chemical dealloying, thermal dealloying, ion beam irradiation and laser drilling while leaving the high strain regions of the stent framework unaffected.

19. The method of claim 15 further comprising disposing a coating material on the low strain areas of the stent wherein the coating material is sprayed from a nozzle arranged to direct the spray at the low strain regions of the stent while leaving the high strain regions unaffected.

20. The method of claim 19 further comprising curing the coating material by exposing the coating material to ultraviolet light.
